(19) United States
(12) Patent Application Publication
(10) Pub. No.: US 2026/0287691 A1
(43) Pub. Date: Sep. 24, 2026

(54) SYSTEMS AND METHODS FOR IMAGE GUIDANCE AND DEVICE TRACKING DURING MAGNETIC RESONANCE IMAGING

(71) Applicant: SUNNYBROOK RESEARCH INSTITUTE, TORONTO (CA)

(72) Inventors: JAYKUMAR H. PATEL, NORTH YORK (CA); GRAHAM A. WRIGHT, TORONTO (CA)

(21) Appl. No.: 19/148,723

(22) PCT Filed: Jun. 23, 2023
(86) PCT No.: PCT/CA2023/050881

§ 371 Date: Jul. 17, 2025

Related U.S. Application Data
(60) Provisional application No. 63/439,399, filed on Jan. 17, 2023.

(51) Int. Cl. G01R 33/28; G01R 33/48; G01R 33/58

(52) U.S. Cl. CPC G01R 33/287; G01R 33/4826; G01R 33/583

(57) ABSTRACT

Systems and methods are provided for tracking an interventional device during a magnetic resonance imaging procedure. A set of time-varying 3D non-Cartesian k-space trajectories are employed to collect an undersampled image dataset. The undersampled image dataset is processed to detect a local high-signal region having a known location on or within the interventional device, such as the location of a tracking coil, and employed to provide feedback for tracking a position of the device. At least some of the undersampled image dataset may be collected with a set of imaging coils and processed to generate an anatomical image. Image data from multiple datasets may be processed to generate a higher-resolution anatomical image, and may optionally be further processed to generate a visualization of the device, which may be combined with the anatomical image. The tracked position of the device may be employed to facilitate motion correction of the anatomical images.

### CROSS-REFERENCE TO RELATED APPLICATION

This application claims priority to U.S. Provisional Patent Application No. 63/439,399, titled “SYSTEMS AND METHODS FOR IMAGE GUIDANCE AND DEVICE TRACKING DURING MAGNETIC RESONANCE IMAGING” and filed on Jan. 17, 2023, the entire contents of which is incorporated herein by reference.

### BACKGROUND

The present disclosure relates to image-guided medical procedures involving magnetic resonance imaging.

The utilization of minimally-invasive processes is increasing across the globe for managing and curing a wide range of diseases. These treatments are designed to diminish the intricacy and cost of more invasive procedures, for example, cardiac minimally-invasive interventions are preferred over open-heart surgery. During these endovascular procedures, a medical device is inserted into the body via vessels such as the peripheral arteries or veins. A small incision is created to access the vessels, and a sheath is inserted to the desired area before the interventional device is introduced over the guidewire. Guiding the device properly to the desired area is difficult. For this reason, X-ray, computed tomography (CT), ultrasound, magnetic resonance (MR) imaging, or a combination of these modalities, can be used to generate real-time images to guide the device to the necessary region.

Magnetic resonance imaging (MRI) is commonly used to diagnose diseases and monitor their progress. Interventional approaches, such as radiofrequency or cryo-ablations, stent placements, or angiography, biopsy are mostly done with CT, ultrasound, and x-ray fluoroscopy; however, recent improvements in MR software and hardware have enabled MR image-guided procedures, which can be entirely completed with the help of MR imaging, allowing visualizing and tracking of instruments and devices, to the actual therapy. The number of minimally-invasive interventions done with MRI is increasing, as it can provide soft tissue contrast with two-dimensional and three-dimensional images with quantitative, qualitative, functional and structural information to direct the treatments without exposing the patients or clinicians to unnecessary radiation.

Image guided procedures greatly benefit from the ability to intraoperatively track interventional devices, enabling clinicians, physicians, and radiologists to maneuver interventional devices to the desired area with image-guided assistance. While various technologies exist for the intraoperative tracking of interventional medical devices, the tracking of interventional medical devices that are used during an MR-guided procedure present a unique set of challenges. Current approaches to the tracking or monitoring of MR-compatible interventional devices during MR-guided procedures employ either passive or active methods. Passive methods take advantage of the intrinsic physical properties of the devices to create disparities between the device and the encompassing tissue seen using traditional MR scans. Passive tracking can generate either positive or negative contrast due to paramagnetic or ferromagnetic markers, intravascular contrast agents or specialized substances (e.g. nanoparticles). The precision and accuracy of the navigation depends heavily on the MR sequences, which can be either 2D or 3D. 2D imaging offers real-time passive monitoring while 3D imaging generates the image within a few seconds, limiting the ability to accurately guide the devices.

Active tracking techniques, on the other hand, make use of the incorporated solenoidal receiving micro-coils within the interventional devices which act as a tracking coil, tuned to the surrounding protons (blood or tissue). An active tracking coil is utilized to monitor devices within MRI, which has a greater signal-to-noise ratio (SNR) and more accurate spatial and temporal resolution than passive techniques. This is accomplished by stimulating the protons and applying gradients in the x, y, and z directions to capture signals along a linear path in k-space (e.g. using rectilinear/Cartesian or radial trajectories) to identify the tracking coils. The linear signal in k-space yields a sharp peak at the location of the tracking coil in the MRI reference, and algorithms/systems interpret the signal close to the peak to provide the tracked coil location.

### SUMMARY

Systems and methods are provided for tracking an interventional device during a magnetic resonance imaging procedure. A set of time-varying 3D non-Cartesian k-space trajectories are employed to collect an undersampled image dataset. The undersampled image dataset is processed to detect a local high-signal region having a known location on or within the interventional device, such as the location of a tracking coil, and employed to provide feedback for tracking a position of the device. At least some of the undersampled image dataset may be collected with a set of imaging coils and processed to generate an anatomical image. Image data from multiple datasets may be processed to generate a higher-resolution anatomical image, and may optionally be further processed to generate a visualization of the device, which may be combined with the anatomical image. The tracked position of the device may be employed to facilitate motion correction of the anatomical images.

Accordingly, in a first aspect, there is provided a method of tracking an interventional device during a magnetic resonance imaging procedure, the method comprising:
(a) employing a set of time-varying 3D non-Cartesian k-space trajectories to collect an undersampled magnetic resonance imaging dataset;
(b) processing the undersampled magnetic resonance imaging dataset to detect a local high-signal region having a known location on or within the interventional device; and
(c) employing the detected local high-signal region and the known location to provide feedback for tracking a position of the interventional device.

In some example implementations of the method, at least a portion of the undersampled magnetic resonance imaging dataset is collected using a set of imaging coils, the method further comprising processing the portion of the undersampled magnetic resonance imaging dataset collected with the set of imaging coils to generate an anatomical image. The method may further include employing tracked position of the interventional device to perform motion correction on the anatomical image.

In some example implementations, the method further comprises, after having performed steps (a)-(c) a plurality of times, thereby obtaining a set of undersampled magnetic resonance imaging datasets;
(d) processing the set of undersampled magnetic resonance imaging datasets to generate an anatomical image.

In some example implementations, the method further comprises employing the tracked position of the interventional device to perform motion correction on the anatomical image.

In some example implementations, the method further comprises performing step (d) one or more times, such that the anatomical image is updated less frequently than the feedback associated with the tracked position of the interventional device.

In some example implementations, the method further comprises employing the set of time-varying 3D non-Cartesian k-space trajectories to generate visualization of the interventional device. The method may include generating a composite image comprising the anatomical image and the visualization.

In some example implementations of the method, the set of time-varying 3D non-Cartesian k-space trajectories fully sample k-space.

In some example implementations of the method, the interventional device comprises a catheter.

In some example implementations of the method, each set of time-varying 3D non-Cartesian k-space trajectories is collected at a different cardiac cycle.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 is a schematic representation of the method for device tracking for time-varying gradient trajectories.

FIG. 2 is a schematic representation of the method for acquiring the time-varying gradient data for simultaneous device tracking and for anatomy visualization. The figure shows an example of the method for cardiac interventions.

FIG. 3 is a schematic representation of a method for acquiring the time-varying gradient data for device tracking and for motion-correction of the undersampled anatomical imaging.

FIG. 4 is a schematic representation of the method of motion-correction of the fully-sampled anatomical imaging with use of device tracking surrogate information for motion-correcting data from time-varying gradient trajectories.

FIG. 5 illustrates a temporal snapshot of the time-varying gradient trajectories for active device tracking.

FIG. 6 illustrates a temporal snapshot of the time-varying gradient trajectories for undersampled anatomical imaging.

FIG. 7 is a schematic representation of the method for an example pipeline of the device tracking, motion-correction of the undersampled and fully-sampled anatomical imaging, and device shaft visualization for a cardiac intervention.

FIG. 8 shows the accuracy of the positions and orientations for the phantom study for the stationary and respiratory simulated motion for the device tracking.

FIG. 9 shows the accuracy of the device tracking and device size measurement in comparison to the prior known geometry of the devices in an in-situ setting.

FIG. 10 shows the result of the device visualization in 3D within an in-situ setting.

FIG. 11 shows the result of the device visualization in 3D within an in-vivo setting.

FIG. 12 shows the accuracy of the positions and orientations for the human study for device tracking during respiratory motion.

FIG. 13 schematically illustrates an example system for performing device tracking and anatomical imaging based on time-varying gradient trajectories.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately” are meant to cover variations that may exist in the upper and lower limits of the ranges of values, such as variations in properties, parameters, and dimensions. Unless otherwise specified, the terms “about” and “approximately” mean plus or minus 25 percent or less.

It is to be understood that unless otherwise specified, any specified range or group is as a shorthand way of referring to each and every member of a range or group individually, as well as each and every possible sub-range or sub-group encompassed therein and similarly with respect to any sub-ranges or sub-groups therein.

Unless otherwise specified, the present disclosure relates to and explicitly incorporates each and every specific member and combination of sub-ranges or sub-groups.

As used herein, the term “on the order of”, when used in conjunction with a quantity or parameter, refers to a range spanning approximately one tenth to ten times the stated quantity or parameter.

Unless defined otherwise, all technical and scientific terms used herein are intended to have the same meaning as commonly understood to one of ordinary skill in the art. Unless otherwise indicated, such as through context, as used herein, the following terms are intended to have the following meanings:

As used herein, a Cartesian k-space trajectory is a data acquisition method utilizing phase encoding gradients and frequency-encoding readouts to form a rectilinear grid layout in k-space. The phase encoding gradients are responsible for recording the location of nuclear spins along a specific axis, while the frequency-encoding readout is used to acquire multiple points in the k-space during a predefined temporal window. Examples of Cartesian k-space trajectories include 2D and 3D cartesian trajectories, and Echo planar imaging.

As used herein, a non-Cartesian k-space trajectory is a data acquisition scheme which uses non-rectilinear and/or non-uniform sampling patterns. Pseudo-Cartesian (hybrid) trajectories are a mix of Cartesian and non-Cartesian approaches, such as using non-Cartesian sampling on a Cartesian grid or sampling non-Cartesian paths in the kx-ky plane with Cartesian sampling along the kz dimension, or any other combination thereof. Examples of non-Cartesian k-space trajectories include two-dimensional spirals, radial spokes and a range of variations. Three-dimensional trajectories include stack of spirals (a hybrid of two-dimensional spirals in kx and ky with the spirals repeated at each kz position), shells, spherical stack of spirals and more. It is important to note that some non-Cartesian k-space trajectories have temporally invariant gradients during the readout, while others are subject to time-varying gradient trajectories, allowing for complex data acquisition schemes.

As used herein, a time-varying gradient k-space trajectory is a sampling pattern where the gradient amplitude changes during the readout. Examples of non-time-varying gradient trajectories include Cartesian, radial, and PROPELLER acquisitions. Time-varying gradient trajectories encode the spatial frequencies in k-space by varying the gradient amplitude and direction causing non-linear path in the k-space. Examples of non-Cartesian time-varying k-space 2D trajectories include spirals, rosettes, concentric rings and many more variations. 3D time-varying gradient trajectories include but not limited to, Stack of Spirals, Shells, Spherical stack of spirals, Yarn and Spiral-projection.

As used herein, the phrase “anatomical roadmap” refers to an image or a slice or multiple slices or volume or volumes of the anatomy to help during the procedure or procedures to guide or navigate or treatment planning or diagnostic or reference or depict structural landmarks or used in any other manner to guide or draw any conclusions during the produce.

As described above, MRI has the potential to revolutionize interventional procedures, provided that 3D imaging of anatomy can be supplemented with intraoperative tracking of interventional devices. While both passive and active approaches have been developed for the tracking of MR-compatible interventional devices during an MR-guided procedure, active tracking methods have shown the greatest promise.

The conventional approach to tracking active devices utilizes a temporally interleaved approach, where separate MR sequences are used to obtain three or more perpendicular or diagonal projections of the actively-tracked device. A 1D Fast Fourier Transformation (FFT) is then applied to the projections to determine the position [x, y, z] of the device. The physical position of the device may then be employed to layer the anatomical images for displaying the device location in relation to the anatomy. This conventional approach to active tracking necessitates separate imaging and tracking pulse sequences, rendering it inadequate in the presence of motion. The present inventors sought out to develop an improved approach that could facilitate near-simultaneous anatomical imaging and tracking during MR-guided procedures.

The present inventors carefully considered the impact of k-space trajectory selection on the ability to perform rapid and real-time tracking of an interventional device. During MR imaging, the magnetic field gradients spatially encode the signal and define the trajectory to fill the k-space. A trajectory is defined as the path along which the spatial encoding data is acquired in the k-space. MR sequences use gradients (Gx, Gy, Gz) to guide the trajectory to the desired k-space position to acquire the data, conventionally usually in a Cartesian grid. The trajectory of a readout is determined by the gradients utilized.

Conventional Cartesian trajectories acquire data along a frequency encoding direction, where a row is first collected before going to the next row, which is the phase encoding direction, and during the acquisition process, the gradients are constant. Non-Cartesian trajectories follow non-traditional k-space paths. For example, a 2D radial trajectory acquires the data with spokes at different polar (and azimuthal, in the case of 3D radial trajectories) angles, using temporally static gradients.

A prior attempt to achieve combined anatomical imaging and interventional device tracking employed 3D radial projection-based sequences, in which spokes passing through the center of k-space are acquired at arbitrary polar and azimuthal angles. Three perpendicular radial projections are then utilized to locate the device through a 1D FFT, while data simultaneously acquired from the imaging coils is used for anatomical imaging. This prior work has shown that acquiring three or more trajectories is sufficient to localize the device. Although radial (spoke) k-space trajectories are non-Cartesian, they are nonetheless time-invariant, with each trajectory inefficiently sampling only a small region of k-space.

This inefficient sampling of k-space with each trajectory limits the utility of such trajectories for combined interventional device tracking and anatomical imaging. Indeed, the present inventors realized that a significant improvement could be achieved by employing time-varying, non-Cartesian k-space trajectories for device localization and anatomical imaging. The present inventors realized that such an approach could facilitate improved device localization while also enabling integrated anatomical imaging and tracking during MR-guided procedures.

Time-varying 3D k-space gradients allow for high-SNR sequences that efficiently sample k-space. Such an approach facilitates highly undersampled imaging in multiple dimensions, such as time along the cardiac cycle, time along the respiratory cycle, T1 values, T2 values, T2* values, and echo time. Furthermore, time-varying trajectories are more robust to motion and flow artifacts, which is necessary for interventional procedures, particularly in the case of cardiac anatomy.

One example of a time-varying, non-Cartesian 3D trajectory is a “3D cone” trajectory. A 3D cone trajectory starts from the center of k-space and spirals outwards to create a cone-like path in 3D space, acquires data along a curved, non-linear k-space trajectory that follows the surface of a cone. Additional examples of time-varying, non-Cartesian 3D k-space trajectories include “stack of spirals”, shells, “spherical stack of spirals”, yarn, and spiral-projection.

The present inventors also realized that the conventional 1D projection-based reconstruction techniques, which were employed in conventional approaches to active device localization, and also employed in the aforementioned radial spoke trajectory based approach, are not feasible for time-varying k-space gradients. Indeed, traditional projection-based active device tracking techniques are inadequate for such nonlinear, nonplanar, and nonuniform k-space trajectories, thus limiting the use of time-varying gradient trajectories for MRI-guided procedures.

In particular, time-varying trajectory methods of localization cannot be performed with existing methods, such as methods that determine the peaks of the signal or determine the centroid around the peak. These methods assume Δf=γ [symbol] ·Δ [symbol] , where for time-varying gradient the [symbol] has a time varying component, making it a much more difficult problem to solve with existing methods,

$$Δ f = {\int }_{0}^{t} γ \vec{G ( τ )} \cdot Δ \vec{r ( τ )} d τ .$$

Accordingly, as described in further detail below, the present inventors developed an improved approach to device localization that leverages a common pulse sequence for tracking and anatomical imaging, based on time-varying, non-Cartesian k-space trajectories. Various example embodiments of the present disclosure employ the use of time-dependent 3D gradient trajectories during an MRI-guided/aided intervention to facilitate the simultaneous visualization and position tracking of an interventional MR device, with optional precise motion correction of the under-sampled and well-sampled datasets, based on the device-tracked positions.

The embodiments of the present disclosure may be beneficial in achieving accurate and near real-time device tracking that enables simultaneous anatomical imaging and device visualization. Moreover, as described further below, the example embodiments of the present disclosure may facilitate one or more additional benefits or functions, such, as, but not limited to, the generation of anatomical roadmaps to target the interested regions, motion correction of the interventional MR-compatible device, and accurate assessment of the treatment endpoints.

In some example embodiments, systems and methods are provided that employ the use of the highly undersampled image data collected via a time-varying gradient to locate and track an MR-compatible interventional device, based on detection of a high-intensity signal associated with a local region of the interventional device.

In other embodiments, the present disclosure provides systems and methods that employ the use of a time-varying gradient trajectory for device localization to track the motion of the anatomy in localized spots (or regions) to facilitate motion-resolved images to improve device visualization locally.

In other example embodiments, systems and methods are disclosed for generating an image or image annotation that facilitates visualization of the interventional device, with the time-varying gradient trajectories being employed for both active device location tracking and for visualization imaging of the device.

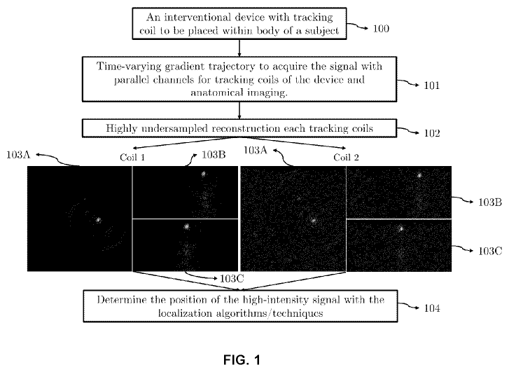

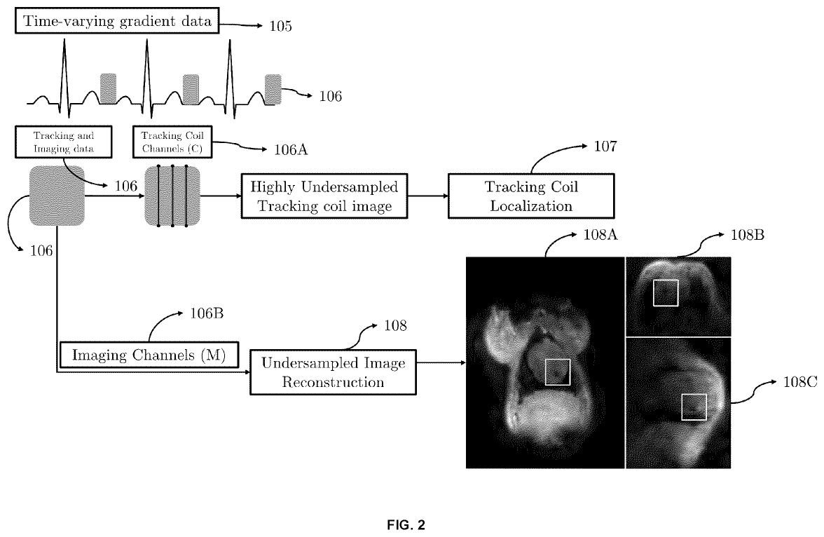

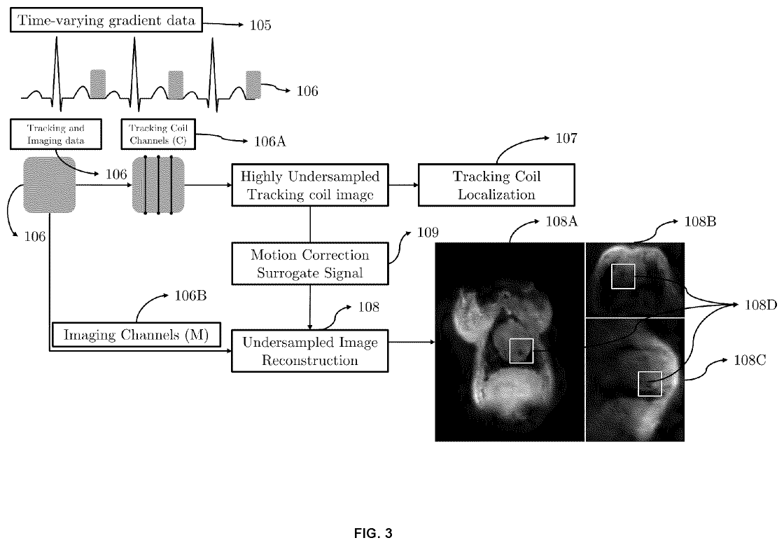

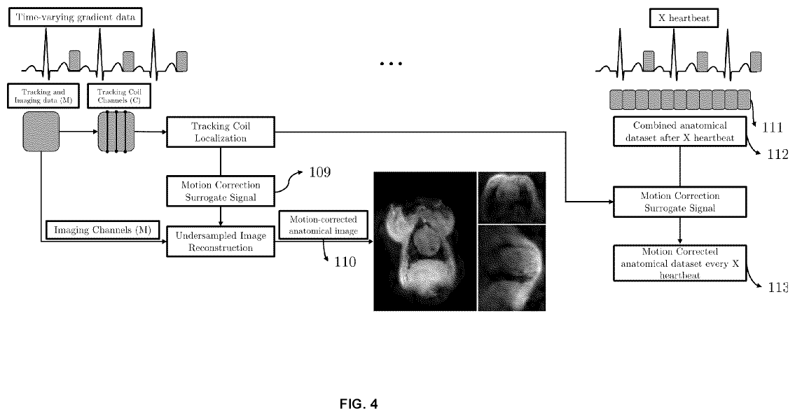

Several example workflows of the present disclosure are schematically illustrated in FIGS. 1-4. FIG. 1 illustrates an example method of locating device tracking coils using the reconstructed volume with very highly undersampled data. FIG. 2 presents an example method for acquiring the time-varying gradient data for simultaneous device tracking and anatomy visualization. FIG. 3 illustrates an example method of employing the tracked positions of the interventional device for correcting motion in the undersampled anatomical data. FIG. 4 illustrates the use of the tracked position of the device for correcting motion in the fully sampled anatomical data (or near fully sample anatomical data) to gain the structural images for guidance and to employ device visualization using tracked positions of the devices. These example workflows, and additional example workflows, are described in detail below.

FIG. 1 shows an example method of device localization using undersampled 3D time-varying gradient trajectories. The initial step 100 involves the acquisition of data through the utilization of a 3D time-varying gradient trajectory (sequence) 101,102. Each trajectory is employed to sample a distinct subregion of k-space for a given (e.g. assigned) spatial resolution.

The undersampled images of the device tracking coil can be reconstructed with a set of N trajectories, as demonstrated in 103. This reconstruction can be achieved, for example, through gridding reconstruction with the help of an adjoint nonuniform fast Fourier transform (NUFFT). m=NUFFT^{H}(Wy_{c})=F^{H}P^{H}Wy_{c}. The m references to the reconstructed undersampled image, H denotes the matrix adjoint, F is the Fast Fourier transformation operator, P is the trajectory sampling operator that projects the Cartesian data in k-space to the corresponding locations given by the trajectory acquired in the k-space data y_{c} from the tracking coils, and W is the density compensation function (DCF) used to calibrate the intensity of the image by weighting the acquired non-Cartesian data by its relative abundance in k-space. K-space from the tracking coil is utilized as an initial input for the NUFFT reconstruction combined with the pertinent trajectories and DCF. Other reconstruction methods like parallel imaging and/or compressed sensing methods or Artificial Intelligence (AI) image reconstruction can be used reconstruct highly undersampled datasets. Furthermore, there are additional methods to perform undersampled reconstruction of the time-varying gradient trajectories that balance the tradeoff between reconstruction time and image quality.

The number of trajectories N to acquire in each measurement can be determined based on the degrees of freedom. To find the position within 3D space, a minimum of three trajectories following straight lines are required to solve for the position; similarly, two straight-line trajectories are required to solve for the position in 2D space. With the utilization of time-varying gradient trajectories, high intensity points can be identified in undersampled images with a reduced amount of data: a balance between the capability to localize high-intensity points in an image space with respect to aliasing artifacts and the quality of the undersampled reconstruction is considered when selecting the number of trajectories or k-space points.

For example, if the undersampled image of the high intensity point from k-space data of 1 or less trajectories is sufficient for accurate localization, then N<1 can be used for device localization. N<1 is when partial points are acquired/used within a readout to localize the device. Undersampling of the k-space can be performed within each readout and with number of trajectories in any form. Undersampling schemes can be performed prospectively (during the sequence acquisition) or retrospectively (manual undersampling). Generally, more trajectories are needed to reduce artifact prevalence and accurately identify the high intensity point corresponding to the active catheter. The number of trajectories for localizing the high intensity point can also be decided in accordance with the temporal window which is subject to the application.

For a cardiac intervention, the rate at which device position is updated is dependent on N. To increase the frequency of updates, the number of trajectories used to acquire the position is decreased. The value of N can be dynamic, depending on the application or area of navigation or any computational limitations. Generally, for a cardiac intervention, N trajectories can be selected within a diastolic window to minimize cardiac motion, a sliding window can be utilized to track the device continuously or a temporal window can be set up at every X heartbeats, where X is the number of heartbeats. Any other temporal window can be employed, as long as precise localization is achievable.

A localization algorithm can then be employed to determine the location of the tracking coil, as shown in FIG. 1, 104. As the signal of the tracking coil is sparse, the image can be constructed without any significant aliasing. For instance, in a device with multiple tracking coils, individual tracking coil images can be created to locate the tracking coils, as shown in 103A, 103B and 103C. After computing the positions of the tracking coils, the orientation can be calculated for the device.

FIG. 1 outlines an example process for localizing a device. The time-varying gradient trajectory raw data is split into y=y_{c}⊗yₘ, decomposing the data into the imaging yₘ and device tracking y_{c} components by phase array channel number, a combination of the receiver coils placed near to the anatomy of interest which act as (fairly) independent receivers of MRI data. With the data from the device tracking, highly undersampled reconstruction is performed to determine the position of the device with a localization algorithm. There are multiple ways to set the number of trajectories, such as continuously localizing the device with a set of N trajectories and shifting the window by X trajectories or setting a temporal window. The frequency of the device localization may be governed by the size of the shifting window X, the number of trajectories used for localization, computation latency, or the localization algorithms.

A specific example implementation of this embodiment is described in further detail in the Examples provided below. In the example implementation described below, an interventional device with two embedded tracking RF coils was employed for the simultaneous localization and volumetric imaging of the heart. To track the device, 8 cone trajectories (N=8) with a bin/segment width of 42 msec were used to reconstruct the 50× undersampled image of an RF tracking coil, to identify the tracking coil locations and orientations. The orientation vectors were calculated based on the positions of the two micro-coils, with the direction towards catheter tip. By utilizing a GPU, the device was localized at a rate of 3 Hz (3 tip localizations/see or 6 coil localizations/sec). With the implementation of faster and more effective localization algorithms, the frequency of localizing the device can be increased.

In general, to locate the high-intensity bright point in the 3D space, N trajectories can be employed given that N≥3, with the possibility of aliasing artifacts depending on the resolution of the volume and trajectory. It is possible to enhance the highly undersampled reconstruction of the volume for localization of high-intensity by oversampling the center of the k-space with time-varying gradients.

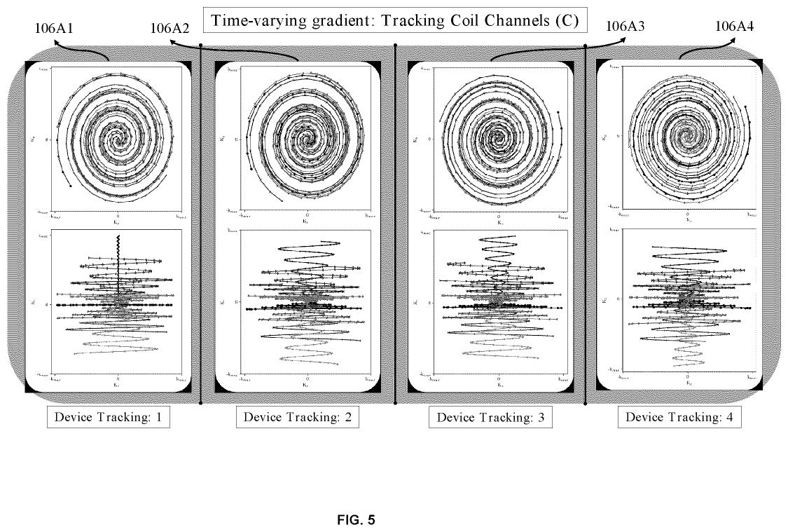

FIG. 5 present the illustrations to show the use of time-varying gradient trajectories to intermittently localize the interventional device in 3D cones. The figure shows data collected during a 200-msec cardiac diastole window, which included imaging (yₘ) and tracking coil (y_{c}, c=2) datasets. These two tracking coil datasets were divided into four equal subsets, each consisting of eight distinct k-space trajectories (106A1, 106A2, 106A3, 106A4). Device localization (FIG. 1) was then executed on each subset to monitor the device's position via time-varying gradient 3D cone trajectories.

Localization of the device from the image can be carried out with a variety of image analysis strategies (including but not limited to image filters, noise removal, image enhancement, thresholding, clustering and edge detection), computer vision techniques (including but not limited to segmentation and object recognition) and signal processing approaches (including but not limited to reconstruction, filtering, wavelet-based domain transfer, and total variation). While this example embodiment is shows an example in which the position locations of the locally high-intensity signals with highly undersampled time-varying trajectory can be extracted using the center of mass localization algorithm, many other techniques can be used for image-based active device localization. These include, but are not limited to, center of mass, template matching, peak detection algorithms, mathematical morphology algorithms, gradient based methods, clustering algorithms or AI models (such as object detections, peak detection).

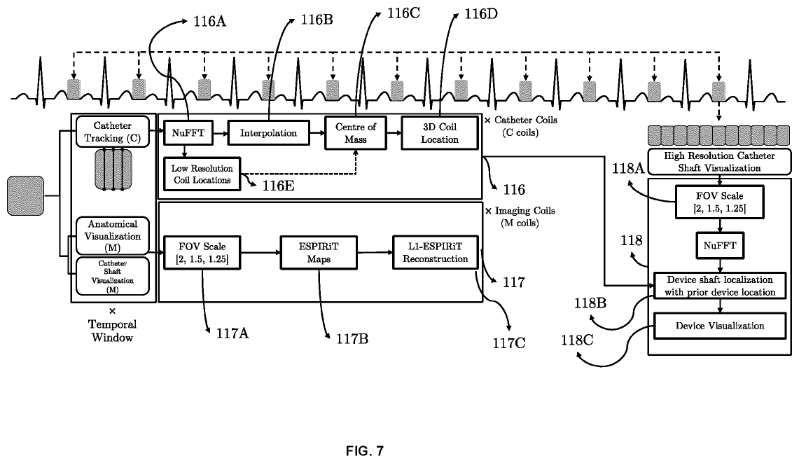

In FIG. 7, box 116 illustrates the device localization algorithm with the use of a 3D cone dataset. The input of the localization algorithm comprises of the raw k-space data (y_{c}) from the device coils channels along with corresponding trajectories and DCFs. The algorithm can be applied to each coil's undersampled images in parallel. NUFFT image reconstruction is first employed to generate the undersampled images. The location of the maximum intensity point is used as a reference point (R) for identifying the high intensity point in the image. To localize the center of the coil in each channel, the undersampled images are interpolated to a high spatial resolution and a 3D center of mass is calculated within a region of interest centered around R.

By utilizing the localized device coils for each channel, the location of the tip can be extrapolated based on prior geometric knowledge of the device. For instance, if a catheter device has two tracking coils (C1, closer to the tip of the device, and C2, far away from the tip of the device) with a distance between them (D12) and a distance between C1 and the tip of the catheter (D1T) then the tip's coordinates can be calculated using the positions of the coils and the previously known distances between the coils and tip:

$$\vec{T} = \frac{\vec{C 1} + \vec{C 2}}{2} + \frac{\vec{C 2} - \vec{C 1}}{D 12} ( D 1 T + \frac{D 12}{2} ) .$$

If there are multiple coils within the device, more channels must be dedicated for tracking instead of imaging. Nevertheless, each tracked coil or point allows for an estimation of the device shaft's geometry via interpolation or extrapolation, along with the prior device geometric information. As an illustration, multiple tracked coils within a catheter can be interpolated with nonuniform rational B-splines (NURBs) to generate a graphical representation of the object, which in this case is an interventional device. There are various techniques which can be utilized to produce a visualization of the device shape depending on multiple tracked points or the high intensity regions of the device.

In some example embodiments, the time-varying non-Cartesian k-space trajectories that are employed for interventional device localization are also employed for anatomical imaging. Such an example embodiment is shown in FIG. 2, which illustrates a method in which imaging data and device localization data are collected simultaneously from a common pulse sequence.

The same radiofrequency pulse is employed to acquire both imaging and device localization data. This is achieved through the utilization of phased array coils which only act as a receiver of the radiofrequency signal. These coils are arranged into a large array and the individual coils detect the signal based on their spatial proximity. During MRI sequences, each coil separately captures a signal in its vicinity, allowing for individual post-processing of the data from each coil in the array. The data from each coil can be used in any combination. The receiver coils in the phase array can be connected to the embedded device tracking coils or any other aforementioned methods for receiving the signal. With this principle, C number of phase array coils can be repurposed for device localization, while M number of coils can be used for imaging the anatomy. For example, FIG. 2 provides an illustration of the data obtained from the separate phase array channels for the time-varying gradient trajectory that can be processed independently. 106A displays the individual coil data used for the device tracking coils (N=8); thus, the frequency of the device localization is higher in comparison to anatomical imaging. 106B contains the data obtained from all the imaging coils (M) that can be employed to create anatomical roadmaps. 106B can be used, for instance, to obtain 3D cone time-varying gradient trajectory raw data from multiple coil channels from the phase array acquired within a diastole temporal window and, with the use of compressed sensing and parallel imaging, the data can be reconstructed. The data from 106A and 106B can be merged to generate anatomical images featuring bright tracking coil signals that display overlapped device localization information. The image reconstruction of 106B for the anatomical imaging can be performed with any number of trajectories N>1, with a tradeoff between the image quality, spatial and temporal resolution, signal to noise ratio or artifacts, as the criteria may vary based on the application.

It is possible to carry out undersampled anatomical imaging, which can be employed for device motion correction (as explained in further detail below), in parallel with location detection (e.g. navigation) of the interventional device, with almost real-time updates of the anatomy. Anatomical image reconstruction can be performed using any suitable method, including, but not limited to, parallel imaging, compressed sensing, temporal and spatial basis reconstruction, model-based reconstruction, or AI reconstruction.

For example, 3D cone time-varying gradient trajectory undersampled reconstruction of the anatomy can be accomplished via [symbol] -norm efficient Self-Consistent Parallel imaging (L1-ESPIRIT). This image reconstruction method involves eigenvalue decomposition in both k-space and image space to determine the subspace calibration area utilizing multi-phase coil data. The calibration region in k-space is where the low frequency data points are located near the center of k-space. To reconstruct the anatomical image, the inverse problem was solved:

$$\text{?} = arg {min}_{m} \frac{1}{2} {‖ \sqrt{W} PFSm - {y}_{m} ‖}_{2}^{2} + λ {\vert Ψ m \vert}_{1} .$$

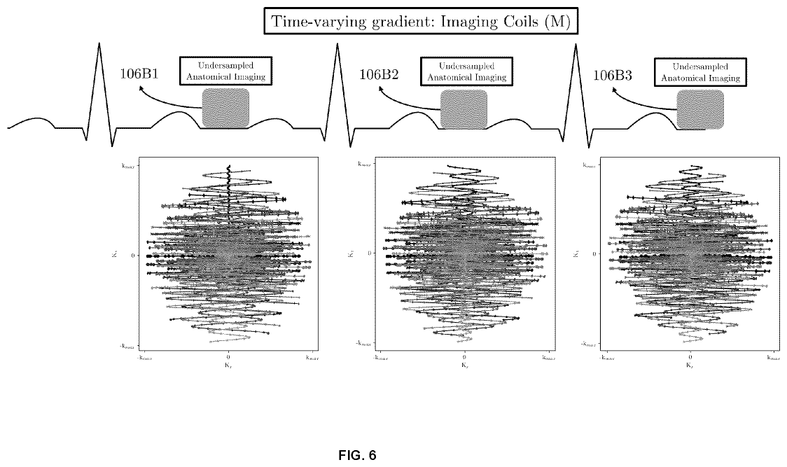

Here [symbol] ∈C^{N}^{ₓ}^{×N}^{_{y}}^{×N}^{_{z}} is the reconstructed anatomical image, F is the Fast Fourier transformation operator, P is the time-varying gradient trajectory sampling operator, and \sqrt{W} is the square root of the DCF for the trajectory sampling. Sensitivity maps Sₘ compute the spatial sensitivity of individual coil for yₘ using the ESPIRIT algorithm. A regularization parameter 2 is used along with the regularization functional, penalizing the l¹ norm of the spatial wavelet transforms Y. The imaging data from all coils are scaled at a higher field of view (FOV) to remove residual aliasing artifacts, and the L1-ESPIRIT reconstruction is performed with corresponding coil sensitivity maps. The anatomy scanning required for interventional techniques can be obtained from the imaging coil in any form of acquisition—whether it be continuous, surrogate triggered, or otherwise—as long as enough data is collected to produce the image with an optimal balance between image quality and other factors. Anatomical images can be produced from a subset of the data, which can be reconstructed using any of the previously mentioned methods for device localization undersampling reconstruction. As demonstrated in FIG. 6, distinctive subsets of the 3D cone trajectories were acquired in a diastolic window, generating anatomical images for each subset with 117, offering the capacity to observe the anatomy over time in the presence of respiratory movement since cardiac motion was eliminated by obtaining the data at a diastolic window where the heart is in its most restful state.

In some example embodiments, the tracked location of the interventional device can be monitored almost in real-time, and the degree of change in its position can be employed as a proxy for movement of the body part that it contacts. Therefore, the tracked position of the interventional device can be employed to generate corrections for counteracting the motion of the anatomy during MR imaging. Such anatomical motion causes motion and flow artifacts in the image, which can significantly reduce the quality of the image and its accuracy for the intervention.

Accordingly, in some example embodiments, the tracked position of the interventional device can thus provide a surrogate signal that can be employed to adjust the motion in the k-space, in a short amount of time, as the motion is recorded within a small portion of the acquisition. Motion of the region of target can be compensated in highly-undersampled anatomical imaging within the image domain, which can lead to sharper, noise-reduced images. This can be done through image registration or denoising algorithms, or any variation or combination of those. The motion can also be corrected within the k-space data, for example with autofocusing or self-navigation motion correction methods.

As both the imaging and device localization data undergo the same motion during the acquisition phase, motion artifacts and blurriness are generated in the image, necessitating motion correction or a rescan which will eventually result in a longer scan and procedure time for MRI-guided interventions. Since device localization is performed with dataset acquired in order of milliseconds the motion within each subset is compared to the anatomical imaging data. Therefore, device localization can be used as a surrogate signal to counteract the motion and reconstruct motion-resolved anatomical imaging.

FIG. 3 illustrates a pipeline for motion correction with the utilization of the device-tracked locations. The device locations (107) serve as a motion proxy and since the imaging data experiences the same motion, the alteration in the device locations magnitude is employed as a rigid motion. The motion correction (109) can be applied to the k-space data yₘ:yₘ′=Kexp(2πi\vec{k}·d\vec{r}), where \vec{k} denotes the location in k-space, d\vec{r} is the inputted displacement vector such as device localization positions, and yₘ′ is the motion-corrected k-space dataset, which is used to perform undersampled reconstruction with the aforementioned undersampled image reconstruction methods. The results of the motion-resolved reconstruction will resemble the images presented in 108 in all orientations, namely 108A: Coronal, 108B: Axial, and 108C: Sagittal. However, the prescribed slices orientation can vary for example, based on the anatomy or diagnostic purposes such as oblique angle or natural axis.

In addition, the highly-undersampled anatomical imaging can be used as real-time updates of the anatomical volumes for precise guidance of the interventional tools to the targeted areas or to further correct high-order temporal motion. The high-order temporal motion is the slow motion within larger temporal window. The highly-undersampled images can thus be employed as an image-based navigator for motion correction. This motion creates a misalignment between the anatomical maps used to guide the device and its actual position. To address this misalignment, low-resolution imaging can be used to adjust for the motion within the region of interest, such as respiratory motion of the heart during cardiac interventions, slippage caused by prostate distortion during prostate interventions, or bulk motion of the patient during brain interventions.

Motion correction utilizing device localization improves the visualization of the anatomical imaging. For example, as shown in FIG. 4, the data (y) acquired with the imaging coils and device tracking coils is obtained within a diastolic temporal window. By combining the k-space data (111) acquired from the imaging coils (yₘ) and the number of the heartbeat (H, X∈ [symbol] ), a fully-sampled dataset with a specified spatial resolution can be acquired. In FIG. 4, data was acquired using cardiac triggering, however, various methods for data acquisition exist. The fully-sampled dataset is only one example, and any undersampled or fully-sampled data can be used to generate images for the anatomical roadmaps.

Motion artifacts typically degrade the high-resolution dataset due to the time-consuming process of data acquiring in the presence of motion, resulting into poor anatomical roadmaps. The motion-corrected fully-sampled or partially sampled dataset can be generated using the motion information from the device localization methods. The motion correction pipeline for the fully to partially sampled dataset is similar to the pipeline illustrated in FIG. 3 for undersampled datasets. Initially, for the motion correction, a subset of the k-space data from the imaging coil is combined to generate a larger k-space dataset with corresponding trajectories and DCFs. The device localization positions are used as a proxy for motion by utilizing the corresponding trajectories to calculate the device's position. For example, if 1:N trajectories (y_{c}) are used to compute device positions, then 1:N trajectories in the imaging coil (yₘ) motion proxy will be the change in magnitude of the device position relative to the reference point 113. This way, the motion surrogate signal corresponds to the anatomical imaging dataset as the device and anatomy experience the same motion once the device is located and touching the wall of the organ. The motion correction is performed with aforementioned methods in section 0067 as undersampled motion correction with the device localization. The motion-resolved fully-sampled or partially-sampled dataset can be used as an anatomical roadmap and for accurately visualizing the device shaft within the anatomy (as described above).

It will be understood there are many different methods to monitor the device location and anatomy based on the use of time-varying 3D gradient trajectories, with the methods disclosed herein providing non-limiting illustrative examples. The selection of a suitable method may depend on aspects such as anatomy region of interest, the type of time-varying 3D gradient trajectories, the type of localization algorithms and reconstruction techniques. For example, data associated with time-varying 3D gradient trajectories can be obtained within a restricted temporal window activated by a physiological cue such as an electrocardiogram or respiratory signal. In other example implementations, such data can be continuously acquired over a set period of time. Reconstruction of the localization volume can be performed, for example, based on a selected balance between speed and precision of the tracking. Non-limiting example reconstruction methods include NUFFT, Compressed Sensing, Parallel Imaging Reconstruction, Temporal and Spatial Basis Reconstruction to compensate for the redundancies, and AI-based reconstruction.

High-resolution anatomical imaging can be achieved by combining undersampled anatomical imaging segments, as depicted in FIG. 6, based on the same time-varying 3D k-space pulse sequences used for the device tracking data. FIG. 6 demonstrates how the k-space data is captured at each heartbeat within a diastole. For an assigned spatiotemporal resolution or FOV of the volume/slice or slices, a definite number of readouts (Q) is needed to fill k-space in order to meet the Nyquist criteria for the time-varying gradient trajectories. As shown in FIG. 6, a subset of the Q is gathered within the diastole window (106B1, 106B2, 106B3), and by combining these subsets of Q, a fully sampled reconstruction of the image can be generated. Here any number of subsets can be used to generate anatomical imaging or device localization; subset of Q can be Q/1, Q/2, Q/3, Q/4, Q/1.1, Q/1000, Q/π . . . Q/A, where A∈ [symbol] . FIG. 6 only shows one method in which the data can be acquired however, many forms and combination can be used together to generate image for varies application like device localization, device shaft visualization, anatomical visualization etc.

Each of the undersampled anatomical imaging segments acquires a distinctive set of trajectories, and a complete set that fills the k-space can be acquired to meet the Nyquist theorem. The set of fully sampled trajectories may be repeated once the k-space grid is full. This enables the intermittent collection of a high-resolution fully sampled dataset over a selected temporal collection window (e.g. spanning seconds, minutes, or hours, depending on the desired field of view, resolutions and type of contrasts), which corresponds to the time needed to acquire sufficient data to generate an appropriate image for the corresponding interventional functions. For example, high resolution volume is required to visualize the finer details to perform accurate transeptal crossing during the intervention, whereas during the device navigation it is important to visualize the overall anatomy rather than visualizing the finer details. Therefore, undersampled images are efficient to perform only accurate navigation task but these images do not provide conclusive information about the targets and endpoints of the treatment. To accurately access the treatment endpoint and targeted areas, high resolution images are required and can be acquired or reconstructed based on the information during the procedures. The high-resolution image can be reconstructed when needed using a reconstruction method such as, but not limited to, the example reconstruction methods described above, or variations thereof.

It is noted that while many example implementations of the present disclosure refer to the collection of a set of k-space trajectories that represent a fully-sampled dataset, it will be understood that high-resolution images may be generated based on a dataset that is not completely full. For example, the high-resolution data can be generated from any combination of the subset of Q, it can be Q/1, Q/2, Q/3, Q/4, Q/1.1, Q/1000, Q/π . . . Q/A, where Q is number of readouts required for the fully sampled dataset for a given spatial resolution and FOV and A∈ [symbol] . The Q/A also accounts for the undersampling schemes performed within each readout. Such as, anatomical image can be generated with using every nth point in the readout and/or every nth trajectory readout, n∈I. This includes all the undersampling schemes such as, variable density, random or any percent of data (0≤% of data≤100, 10%, 20%, 30%, 40%, 50%, 60%, 70%, 80%, 90%).

While the aforementioned device position tracking methods can facilitate the localization of one or more local regions on an interventional device, it would nonetheless be beneficial to be able to visualize a continuous portion of the device, in order to be able to provide a clearer visualization of its overall placement and form inside the body, including, for example, its size, its orientation, or other physical characteristics. Indeed, it has been proposed to coat an interventional device with paramagnetic components in order to create a bright signal in MRI sequences. However, a principal drawback with this visualization method is that it relies on coated materials which can be expensive, and depending on the imaging parameters, the MRI images can be overwhelmed by the intensity of the device or create artifacts, making it difficult to make out the anatomy.

Another known method of visualizing or viewing an interventional device (i.e. on a display showing anatomical image data) is via a passive process, the materials of the interventional device creates positive or negative contrast within an image leading to a susceptibility artifacts or a gap in the signal or bright intensity regions making it hard to differentiate between the anatomy and the artifacts. Consequently, it is usually not recommended to visualize an interventional devices as an artifact, since there is a risk that the artifact size varies depending on the type of sequences, applied current within the devices and other factors making the device appear bigger or smaller than the actual size of the device. One of the main drawbacks of the existing visualization technique is that it can be difficult to identify the devices in the anatomy and it all depends on the direction of the device with respect to the primary magnetic field of the MRI.

In many procedures, it can be essential for a clinician to be able to view a real-time (or near-real-time) image showing the interventional device relative to the anatomy, in order to successfully direct the interventional device to the desired area, which can be a complex task due to the intricate anatomy and challenges in maneuvering the device. An example of visualizing devices with a coating and keeping track of the device with projection-based sequences is present in U.S. Pat. No. 8,532,742B.

In contrast to such conventional methods, the time-varying gradient 3D trajectories and undersampled images employed for device localization may be employed to facilitate an intraoperative visualization of the interventional device. Indeed, by utilizing a 3D passive imaging technique along with the device localization information collected as described above, it is possible to segment and generate a visualization of the interventional device, optionally relative to an anatomical image generated based on full-sampled image data.

Passive device imaging can yield a signal void or susceptibility artifact, more generally described as a signal pattern, that helps distinguish the shape of interventional devices. Since the width of these devices is often in the order of millimeters, it is essential to use high-resolution imaging to visualize the shaft. However, the signal pattern generated in passive device imaging can cause confusion between the anatomy and device shaft. This can be addressed by using prior localization information to recognize the key features of the device, acting as a placeholder for the initial search space. Once the location of the device is determined through image processing methods, image filtering, object detection, or segmentation techniques can be employed to overlay the device shaft onto the anatomical roadmaps. For example, 3D cone time-varying gradient trajectories can be utilized to visualize the passive device in partially or fully sampled datasets. The movement of the heart affects the passive device signal pattern in such a way that it blurs the image. To overcome this issue, device locations can be utilized to obtain motion-resolved images, or can use image processing techniques such as weighted total variation denoising to increase edge detection or a gaussian filter to indiscriminately reduce noise. Subsequently, the device shaft can be highlighted from the passive imaging using volumetric ray-tracing with minimum/maximum intensity projections. This latter technique consists of generating a 2D or 3D image by selecting the minimum or maximum intensity voxel along the projection vector. Nevertheless, there are still other ways to highlight the device shaft from passive imaging. Additionally, this method can be applied to positive contrast passive imaging, as it helps reduce the intense signal from the device shaft on the anatomical image, making it easier to delineate features within the anatomy.

In some example implementations, the extracted device visualization and the one or more tracked locations can be overlaid on top of, or integrated with (as a composite image), the low-resolution real-time anatomical images and/or high-resolution anatomical images, for example, for navigation or target purposes.

The preceding example embodiments have described several systems and associated methods that employ the use of time-varying 3D k-space trajectories to facilitate any one or more of (i) location tracking of an interventional device, (ii) rapid low-resolution anatomical images, that are optionally motion-corrected based on location tracking of an interventional device, (iv) intermittent high-resolution anatomical images, that are optionally motion-corrected based on location tracking of an interventional device, generated based the use of a set of time-varying 3D k-space trajectories that provide a fully-sampled image, for example, at a frame rate that is less than that of location tracking of the interventional device, and (v) visualization of the interventional device relative to anatomical image data. While any one or more of these example aspects may be employed in any given implementation, a non-limiting example integrated workflow is illustrated in FIG. 7.

FIG. 7 illustrates an example workflow in which anatomical imaging of the organ, interventional device tracking, and interventional device visualization are all integrated based on a common pulse sequence that employs time-varying 3D gradients (yielding 3D k-space trajectories) to support an MRI-guided intervention.

The image data is acquired by the phase array channel, where M (106B) and C (106A) are the coils devoted to acquiring the imaging and device tracking, respectively. The plurality of coils, M and C, could be combined with the MRI in any suitable configuration.

As explained above, the imaging data collected with coil(s) M (106B) is employed for device visualization and anatomy imaging, while the tracking data collected with coil(s) C (106A) is employed to locate, in an intraoperative frame of reference, the localized high-intensity bright tracking coils (or other suitable local signal generating means), based on the common set of time-varying 3D gradient trajectories.

The tracked location of the interventional device may be employed as a spatial reference to perform motion-correction for the real-time anatomical imaging, as previously described with reference to FIGS. 3 and 4, thereby achieving improved visualization of the device and anatomy.

The process of localizing the device, motion-corrected anatomical visualization, and device shaft visualization is outlined in FIG. 7. The device localization is carried out with pipeline 116 beginning with the device tracking k-space data from the coils C (106A). Each subset of the data utilized for the device localization is reconstructed into an image with NUFFT (as described in 0049) and the maximum pixel of the image is identified to pinpoint the high intensity point in the low-resolution dataset as a preliminary starting point, 116E.

In order to enhance the localization precision of the device, the NUFFT reconstructed images are interpolated to a higher spatial resolution (116B). For instance, if the spatial resolution of the pixel to localize the data is W, then the error in localization using the image is discrete to the spatial resolution of the image. If the spatial resolution of the image is 5 mm, then one pixel error in one direction would lead to 5 mm of error for device localization. Therefore, to precisely pinpoint the localization of the device low spatial resolution dataset were interpolated to high spatial resolution dataset.

The device position (116D) was then determined using the center of mass (116C) in the area nearest to the maximum pixel location from the low-resolution coil localization.

In FIG. 7, 117 illustrates the workflow of the anatomical and low-resolution catheter shaft visualization, utilizing the data from the imaging coil (M, 106B). The k-space data was scaled to larger FOV to remove the aliasing artifact (117A). An ESPIRIT map (117B) was computed from the k-space dataset for each imaging coil to recognize the spatial sensitivity, which helps with the parallel imaging reconstruction. The undersampled anatomical imaging data was reconstructed with parallel imaging and compressed sensing L1-ESPIRIT reconstruction method; the results of the reconstruction are shown in FIGS. 3 and 4 (108). The catheter device can be seen in the undersampled anatomical data in 108 A, B, C, displayed within a squared box (108D). Motion correction of the anatomical data can be implemented using device position information from 116. To visualize the device shaft, a high-resolution dataset is necessary, as demonstrated in 118.

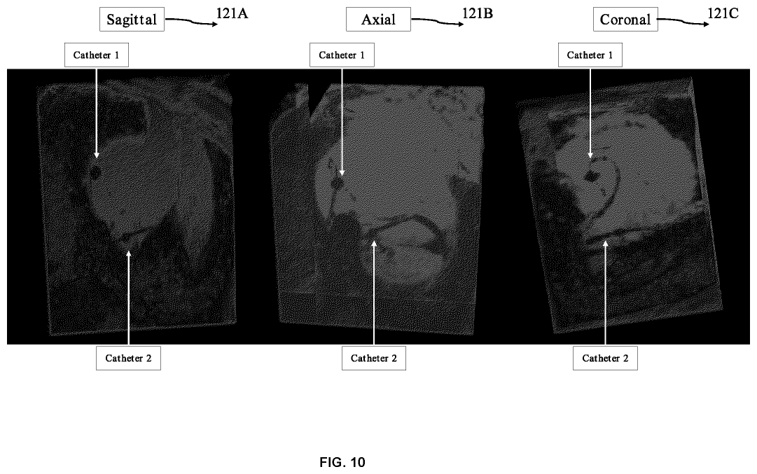

By combining data from the imaging coil(s) (M) over time, either a partially or fully sampled dataset can be used for device shaft visualization. Initially, the combined data is scaled to a larger FOV (118A) similar to 117A and then NUFFT is performed to reconstruct an image(s). With the device position from 116, the device shaft can be highlighted with a minimum intensity projection within a region of interest and, by utilizing prior information about the location and segmentation models, 118B, the highlighted device shaft can be visualized atop the anatomical roadmaps for the interventional procedure, 118C. This is shown in example 2, FIGS. 10 and 11, 121A, 121B, 121C, 122A and 122B.

According to the present example implementation, the device localization signal data from the C tracking coils was acquired during the diastolic temporal window, which was segmented (bins) into N trajectories to achieve the device localization with the highest precision while reducing the number of trajectories and aliasing artifact. The accuracy of the device locations was evaluated by comparing it to the ground truth position and the total acquisition time for N trajectories was close to the conventional projection-based device tracking. For this case, eight trajectories were employed for device localization as this number of trajectories is ideal for providing an aliasing free undersampled volume for accurate localization with a total acquisition time similar to the projection-based methods, which was approximately 42 msec. The number of bins and trajectories within each bin can differ depending on the localization algorithms, time-varying gradient trajectories, data collecting process (continuous or trigger gated) and the type of interventional procedure being performed.

Due to the sparse nature of the signals obtained with the tracked coil, the tracking data can be acquired using little data without aliasing. The localization algorithm can be employed to determine the position of the device based on all the tracking channels C. If there are more than one interventional device within the body each of those devices can be connected together with each device connected to phase array receiver coils in parallel. Each device's tracking coils is allocated an individual phase array coil which would mean more device tracking leads to fewer imaging coils which may affect image qualities, SNR and limit the quality of the undersampled reconstruction.

The same readouts from the device localization from the M imaging coils (yₘ) can be used to reconstruct the undersampled images of the anatomy. The amount of data obtained from the imaging coil(s) can vary based on the application and acquisition scheme. In general, for anatomical imaging, it has been found that N>3 trajectories are needed to perform anatomical imaging (based on an example workflow using 3D cones).

The image representation of the anatomy is not sparse in the image-space domain, meaning there are many pixels with nonzero values due to a wide range of anatomical structures and as a result, more data is required to maintain a balance between the number of trajectories and time to perform highly-undersampled dataset reconstructions. Highly-undersampled reconstruction for a non-sparse signal in the image domain necessitates a slow iterative undersampled reconstruction. For example, 3D cone time-varying gradient trajectory anatomical L1-ESPIRIT reconstruction is performed within couple of seconds with use of a GPU. This can be significantly improved with AI-based reconstruction to achieve close to real-time update of a volume per heartbeat for interventional procedures. This real-time undersampled anatomical data are then reconstructed, optionally with motion-correction from the position(s) acquired from aforementioned device localization methods.

After acquiring image data spanning a set of k-space trajectories that represent a fully-sampled dataset, or a nearly fully-sampled dataset, the image data corresponding to this set may be processed to generate a higher-resolution image. This anatomical image may be employed, for example, to identify the finer structure of the anatomy and a semi real-time updated anatomical roadmap for guidance. In addition, the tracked position of the catheter can be employed, for example, to annotate or modify the anatomical image to identify and/or highlight the device, and optionally to perform motion correction on the image for sharper and improved device visualization.

The anatomical images acquired from the yₘ from M coils can be utilized for numerous objectives during the interventional procedure, contingent upon the contrast (T1-weighted, T2-weighted, T1-weighted with contrast, T2-weighted with contrast, Diffusion-weighted, Magnetic Resonance Angiography, Magnetic Resonance Spectroscopy and much more), image quality (artifacts, poor undersampled reconstructions), SNR, spatiotemporal resolutions and many more. These undersampled anatomical images can also be used for motion-correction as a motion surrogate to generate high-resolution motion-resolved images, which can act as anatomical maps to identify the targets and for treatment evaluation. The information gain from the anatomical roadmaps varies based on an image contrast and resolutions. For example, T1-weighted imaging can be used to visualize the lesions created by ablation catheter, the myocardium infarction can be visible with T1-weighted imaging using intravenous gadolinium contrast, the diffusion-weighted sequences can be used to visualize the movement of water molecules, T2-weighted imaging can be used visualize the edema. Therefore, based on the contrast within an MRI, a wide range of anatomical roadmap can be created while tracking the device to target desired anatomy and assess regions of interest. In one example, the 3D cone trajectory time-varying gradient sequences is a T1-weighted sequence implemented for the cardiac intervention to visualize the anatomy and ablation lesions. However, this sequence can be modified for other image contrasts for accurate assessment of the treatment endpoint. For cardiac ablation T1-weighted sequences can be used to verify lesion placement relative to the target.

Many of the example implementations of the present disclosure refer to the use of local tracking coils to facilitate active tracking of an interventional device. In such an example implementation, this is done by employing the tracking coil as a receiver to pick up signals from protons like blood or tissue within the vicinity.

In other example implementations, material or structure may be employed to generate a locally intense MR signal (i.e. local high-intensity signal region) that is detected by one or more imaging coils that do not reside on the interventional device (i.e. are not co-moving with the interventional device) may be employed for active tracking. Such materials or structures may be passive or active. Non-limiting examples of active structures that are capable of generating a locally intense MR signal (e.g. a local signal enhancement) include embedded radiofrequency coils, antennas (magnetically coupled loop antennas, electrically coupled loopless antennas), sensors, etc. Non-limiting examples of passive materials and structures that are capable of generating a locally intense MR signal (e.g. a local signal enhancement) include ferromagnetic or paramagnetic markers, intravascular contrast agents, specialized materials such as nanoparticles, electric lines within the device, and inductively coupled resonant circuits with no physical wires.

While the several example implementations of the present disclosure refer to active tracking and/or visualization of a catheter, it will be understood that the systems and methods disclosed herein may be implemented with or adapted for use with a wide variety of trackable interventional devices, including, but not limited to, catheters, guidewires, needles, implants, much more.

In some of the non-limiting example implementations presented in the Examples below, anatomical imaging and catheter tracking was performed with a 3D GRE cone sequence, which is beneficial for the MRI-guided intervention due to the lower flip angle to reduce the overall radiofrequency specific absorption rate (SAR) than SSFP sequences. SSFP sequences are often used for the intervention due to their excellent contrast depiction of the myocardial tissue to blood, leading to safety issues due to SAR and local catheter heating. Catheter visualization from the GRE sequences allows for the XRF-like view of the catheter shown in FIG. 10. This provides the interventional electrophysiologist with catheter views similar to those derived from standard clinical approaches using x-ray fluoroscopy. In addition, MRI soft-tissue contrast can be overlaid with catheter tip information, prior 3D anatomical roadmaps and real-time 3D anatomical data acquired in parallel to facilitate the navigation of devices to anatomic targets during the procedures.

Speed is one of the most important considerations for image-guided interventions. The present example methods, which employ time-varying gradient 3D trajectories, efficiently sample the center of the k-space each readout, making them SNR efficient, motion robust and allowing for highly accelerated reconstruction.

Due to the high (e.g. 20×, 30×, 40×, 50× or higher) order of undersampling of the active device tracking data, some noise and residual aliasing artifacts were observed, which can cause localization algorithm failures with jumps in tracked device positions. Accordingly, in some example embodiments, that the device localization algorithm can be improved by enforcing prior device geometry specifications to spatially constraint the device locations or temporal smoothing filters or Kalman filters can be applied to restrain unreasonable jumps during the tracking and may improve precision and reliability.

The geometric dimensions of the physical device can be utilized to enhance the accuracy of the device localization by affirming the prior established distance between the tip and tracking coils of the catheters (C1, C2) such as D1T and D12, the width and length of the device, and many other physical details. By combining the undersampled device localization images from each coil with the localization algorithm that enforces spatial restrictions, the individual coils will be able to function as a collective body to improve the precision and steadiness of the estimated position. For instance, the projection-based localization algorithms use prior information about the physical devices to improve the accuracy such as peak-normed gaussian weight or Joint-Position Optimization algorithm. Algorithms that are similar in nature can be employed for pinpointing high-intensity points in an image for time-varying gradient trajectory. For instance, the center of mass algorithm is used to compute device localization in the examples, where device spatial geometrical information can be imposed. Moreover, temporal smoothing filters, Kalman filters, AI predictive models or many other methods can be used to consolidate predictions and measurement updates in order to estimate the dynamic state of the system and accordingly stabilize the device's localization. This can be achieved by leveraging prior information, such as prior device positions, motion surrogate signals, real-time anatomical volumes, anatomical roadmaps, segmentation models etc. This can facilitate improvement in overall procedures, by decreasing the latency time and/or increasing the update frequency of real-time updates and/or accounting for the variable motion errors during the procedures.

For example, for the cardiac intervention using the time-varying gradient, undersampled anatomical images and device locations are obtained. However, due to computation and latency issues, the anatomical information and device locations are delayed, resulting in a disparity between the observed device location and motion, and the actual position of the device. In addition, motion surrogates such as undersampled anatomical images, respiratory bellow, or cardiac ECG of the patient, combined with device localization and geometrical spatial constraints, can be used. Artificial intelligence predictive models or Kalman filtering methods can then be utilized to estimate the device's location and adjust for the motion during the intervention process. Due to irregular breathing motion during the procedure, the models use dynamic measurements to update the predictive models to help estimate the accurate and stable device localization even when the updated measurements of the systems are not available.

To facilitate fast and accurate navigation, each individual component of the navigation has a tradeoff between the resolution and acceleration factor. In the Examples provided below, device active tracking has been demonstrated with 50× undersampling with zero-filled NUFFT reconstruction at 4.4 mm resolution. Due to the sparse and pronounced signal in the image domain, the location of the micro-coils can be easily extracted. This is not observed with passive catheter visualization because the anatomy is imaged simultaneously resulting in non-sparse signals in the image domain.

In the L1-ESPIRIT reconstruction of the undersampled anatomical images described in the Examples provided below, the passive catheter signal patterns (signal voids or susceptibility artifacts) are regularized as the signal pattern width across the device is approximately 1-2 pixels (9F catheter) therefore, a full, or nearly-full, dataset may be employed to visualize a 3D passive catheter shaft with great details. The catheter visualization can be performed similar to device localization if the device transmission line is coupled with the micro coils inside the interventional device. This way undersampled device localization images can be used to also identify the bright structures within an image and with use of sparsity compressed sensing methods can be used to identify device shaft actively similar to image-based device active tracking localization. As a result, the catheter shaft can also be updated with a similar frame per second as 3D anatomical volumes acquired once per heartbeat. Active transmission line imaging can be performed, and with compressed sensing and curve fitting the shaft visualization can be improved, this was shown with biplanar/triplanar MR projection imaging for 2D/3D catheter visualization.

In the example embodiments that integrate device tracking with anatomical imaging and/or device visualization, the visualization and tracking that are based on a common set of k-space trajectories can be adapted to reduce overall computation time and efficient navigation by using prior pre-procedural datasets and segmentation models within the areas of interest.

The interventional techniques are focused on a particular organ. Consequently, when using the existing knowledge of device locations, organ segmentation models, device geometry, and anatomical roadmaps, the device localization and visualization search can be narrowed down to the relevant region of interest. This will lead to enhanced precision and reduced computation time for the device localization algorithm, visualization of the device shaft, and image reconstruction of the anatomical image for the MRI-guided interventional procedure. For example, the tracked position of the device can be used to pinpoint its location within an organ. Utilizing previously segmented models of the organ, minimum intensity projection or other methods can be employed to delineate the device shaft visualization. The segmentations of the organ can be performed on prior anatomical roadmaps and aligned with the procedural anatomical images using image registration methods. Using the device's location as the starting point, a seed region growing algorithm or other segmentation method such as AI segmentation or thresholding segmentation can be employed to delineate the device from the highly undersampled, partially undersampled, fully-sampled, or any amount of sampled anatomical images to make the device shaft visible. The real-time anatomical images combined with fast motion characterization from the tracked device position can provide improved device shaft visualization and localization within region of interest during MRI-guided intervention.

For a cardiac interventional example, segmentations of the heart can be performed on prior anatomical roadmaps prior to the procedure and aligned with the roadmaps with procedural anatomical image with image registration to bring prior roadmaps within the same coordinate frame of reference as the procedure. The actively tracked device position can be used to initialize the search for the device shaft visualization with for example the seed region growing algorithm which requires a precise initial point for accurate segmentation. The search of device shaft segmentation by minimum intensity projection or maximum intensity projection or any other method to extract device shaft can be performed within a small region of space within the heart using prior heart segmentation models, device geometry and locations. For example, if the device is located within a left ventricle (LV), the segmentation of the LV can be applied to the anatomical images to focus on the search of the device within the small region. This will allow for accurate and fast device visualization and localization from any type of anatomical images. In the example case of tracking a catheter within the heart, the real-time anatomical images every heartbeat combined with fast motion characterization and the tracked position of the catheter can be respiratory motion-resolved to precisely navigate to the targeted area during MRI-guided intervention.

The example embodiments of the present disclosure may be employed for a wide range of magnetic resonance imaging guided interventions. Examples include cardiac ablation procedures for diseases such as ventricular tachycardia or atrial fibrillation, or stem cell delivery to the revascularize the heart, MRI-guided prostate biopsy, breast biopsy, neurosurgery, cell/nanoparticle/drug catheterization delivery and much more.

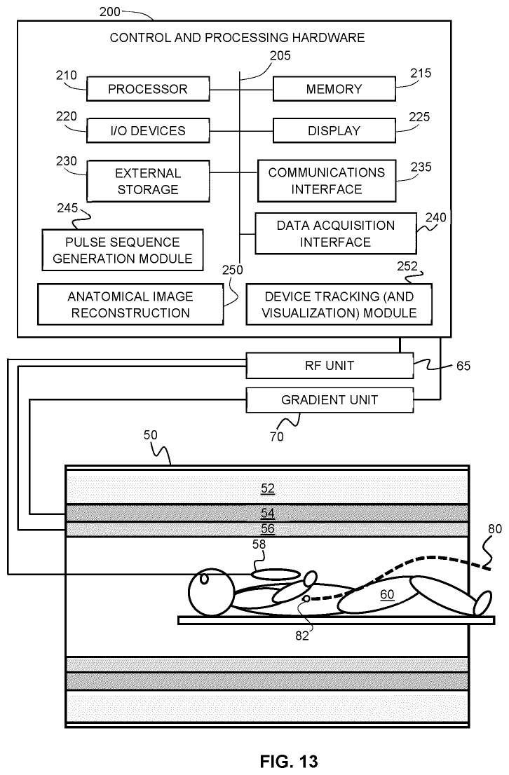

Referring now to FIG. 13, an example system is illustrated for performing dynamic reconstruction with an MRI system according to the example methods described above. The example system includes a magnetic resonance scanner 50 that employs a main magnet 52 to produce a main magnetic field B0, which generates a polarization in a patient 60 or the examined subject. The example system includes gradient coils 54 for generating magnetic field gradients. A receive coil 58 detects RF signals from patient 60. The receive coil 58 can also be used as a transmission coil for the generation of radio frequency (RF) pulses. Alternatively, a body coil 56 may be employed to radiate and/or detect RF pulses. The RF pulses are generated by an RF unit 65, and the magnetic field gradients are generated by a gradient unit 70. An MR-compatible interventional device is shown at 80, having a local signal enhancing region at 82.

It will be understood that the MR system can have additional units or components that are not shown for clarity, such as, but not limited to, additional control or input devices, and additional sensing devices, such as devices for cardiac and/or respiratory gating. Furthermore, the various units can be realized other than in the depicted separation of the individual units. It is possible that the different components are assembled into units or that different units are combined with one another. Various units (depicted as functional units) can be designed as hardware, software or a combination of hardware and software.

In the example system shown in FIG. 13, a control and processing hardware 200 controls the MRI scanner to generate RF pulses according to a suitable pulse sequence. The control and processing hardware 200 is interfaced with the MRI scanner 50 for controlling the acquisition of the received MRI signals. The control and processing hardware 200 acquires the received MRI signals from the RF unit 65 and processes the MRI signals according to the methods described herein in order to perform image reconstruction and generate MRI images.

The control and processing hardware 200 may be programmed with a set of instructions which when executed in the processor causes the system to perform one or more methods described in the present disclosure. For example, as shown in FIG. 13, control and processing hardware 200 may be programmed with instructions in the form of a set of executable image processing modules, such as, but not limited to, a pulse sequence generation module 245 and an image reconstruction module 250. The pulse sequence generation module 245 may be implemented using algorithms known to those skilled in the art for pulse sequence generation, such as those described above.

During MRI scanning, RF data is received from the RF coils 56 and/or 58 (or a local coil residing on or within the interventional device 80). The pulse sequence generation module 245 establishes the sequence of RF pulses and magnetic field gradients for detection along time-varying 3D non-Cartesian k-space trajectories, and MR signals responsively emitted by the patient and detected by the coils are acquired. The anatomical image reconstruction module 250 processes the acquired MRI signals to perform image reconstruction and anatomical image generation according to the example methods described above, or variations thereof. The device tracking (and optional visualization) module 252 processes the acquired MR dataset signals to perform device position tracking and optional intermittent device visualization, according to the example methods described above, or variations thereof.

The control and processing hardware 200 may include, for example, one or more processors 210, memory 215, a system bus 205, one or more input/output devices 220, and a plurality of optional additional devices such as communications interface 235, data acquisition interface 240, display 225, and external storage 230.

It is to be understood that the example system shown in FIG. 13 is illustrative of a non-limiting example embodiment, and is not intended to be limited to the components shown. For example, the system may include one or more additional processors and memory devices. Furthermore, one or more components of control and processing hardware 200 may be provided as an external component that is interfaced to a processing device.

Some aspects of the present disclosure can be embodied, at least in part, in software, which, when executed on a computing system, configures the computing system as a specialty-purpose computing system that is capable of performing the signal processing and noise reduction methods disclosed herein, or variations thereof. That is, the techniques can be carried out in a computer system or other data processing system in response to its processor, such as a microprocessor, CPU or GPU, executing sequences of instructions contained in a memory, such as ROM, volatile RAM, non-volatile memory, cache, magnetic and optical disks, cloud processors, or other remote storage devices. Further, the instructions can be downloaded into a computing device over a data network, such as in a form of a compiled and linked version. Alternatively, the logic to perform the processes as discussed above could be implemented in additional computer and/or machine readable media, such as discrete hardware components as large-scale integrated circuits (LSI's), application-specific integrated circuits (ASIC's), or firmware such as electrically erasable programmable read-only memory (EEPROM's) and field-programmable gate arrays (FPGAs).

A computer readable medium can be used to store software and data which when executed by a data processing system causes the system to perform various methods. The executable software and data can be stored in various places including for example ROM, volatile RAM, non-volatile memory and/or cache. Portions of this software and/or data can be stored in any one of these storage devices. In general, a machine-readable medium includes any mechanism that provides (i.e., stores and/or transmits) information in a form accessible by a machine (e.g., a computer, network device, personal digital assistant, manufacturing tool, any device with a set of one or more processors, etc.).

Examples of computer-readable media include but are not limited to recordable and non-recordable type media such as volatile and non-volatile memory devices, read only memory (ROM), random access memory (RAM), flash memory devices, floppy and other removable disks, magnetic disk storage media, optical storage media (e.g., compact discs (CDs), digital versatile disks (DVDs), etc.), network attached storage, cloud storage, among others. The instructions can be embodied in digital and analog communication links for electrical, optical, acoustical or other forms of propagated signals, such as carrier waves, infrared signals, digital signals, and the like. As used herein, the phrases “computer readable material” and “computer readable storage medium” refer to all computer-readable media, except for a transitory propagating signal per se.

### EXAMPLES

The following examples are presented to enable those skilled in the art to understand and to practice embodiments of the present disclosure. They should not be considered as a limitation on the scope of the disclosure, but merely as being illustrative and representative thereof.

### Experiment 1: Phantom Study

A small container phantom was constructed and filled with a 0.08 mM MnCl₂ solution to mimic the T1 and T2 value of the blood. The solution was created from Manganese (II) chloride tetrahydrate dissolved into distilled water at 23° C. MRI compatible fixtures were used to hold the catheter at a particular location and orientation. An MR-compatible motion stage (MR-1A-XRV2, Shelley Medical Imaging Technologies, London, ON, Canada) was used to allow for precise positioning of the catheter within the bore and to simulate respiratory motion. Respiratory motion was simulated as a sinusoid wave with a displacement amplitude of 20 mm and a period of 4 seconds/cycle. This motion is suspended for ⅓ of the cycle, to simulate end-expiration.

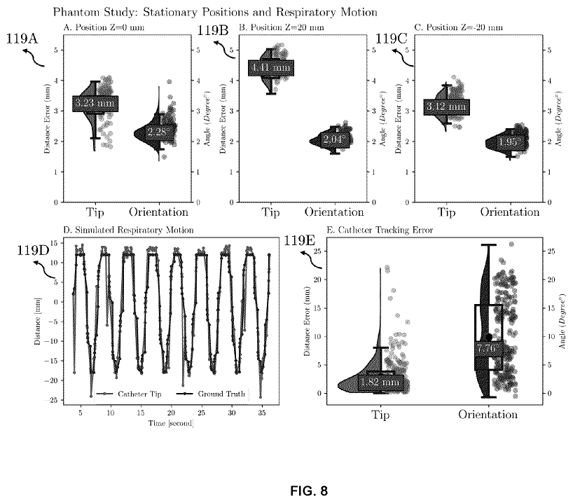

3D active catheter tracking was performed in a static phantom with relaxation properties reflecting blood, and data was post-processed with our GPU-based pipeline. Ground-truth catheter positions were calculated manually from the 2D fast spin echo sequence at different locations from the isocentre and orientations. The mean absolute distance and mean angular error between the ground truth and the observed tip was calculated. The mean absolute error and the mean angular error in tip orientation for the tip position in the phantom study shown in FIG. 8 119A, 119B, 119C. Active tracking was also performed in a dynamic phantom with respiratory motion simulation shown in FIG. 8 119D, 119E. Good agreement between the simulated and tracked motion was observed with 1.82 mm accuracy.

### Experiment 2: In-Situ and In-Vivo Study

The Sunnybrook Research Institute Internal Research Bureau approved all study protocols involving animals. An in-situ and in-vivo study was performed on two 50 kg healthy Yorkshire swine. Animals were given an intramuscular injection of ketamine, and isoflurane gas (1-5%) was continuously delivered through mechanical ventilation during the whole study to maintain anesthesia. For comparison purposes, real-time active catheter tracking during the study was performed with a standard 1D projection-based GRE sequence implemented in RTHawk software. Imaging parameters were FOV=300 mm×300 mm, flip angle=5°, TR=14.3 ms, and tracking rate=23 fps. The position of the catheter was overlaid on a 3D pre-procedural anatomical roadmap with a segmented shell in Vurtigo (Percept, Sunnybrook Research Institute, Toronto, ON, Canada), an interventional MRI guidance software. The catheter was introduced from the femoral artery and with the transseptal crossing, the catheter was placed inside the left ventricles. Once the catheters were placed in the desired area, the custom 3D cone trajectory tracking sequence was used to examine the orientation and position of the catheter within the heart.

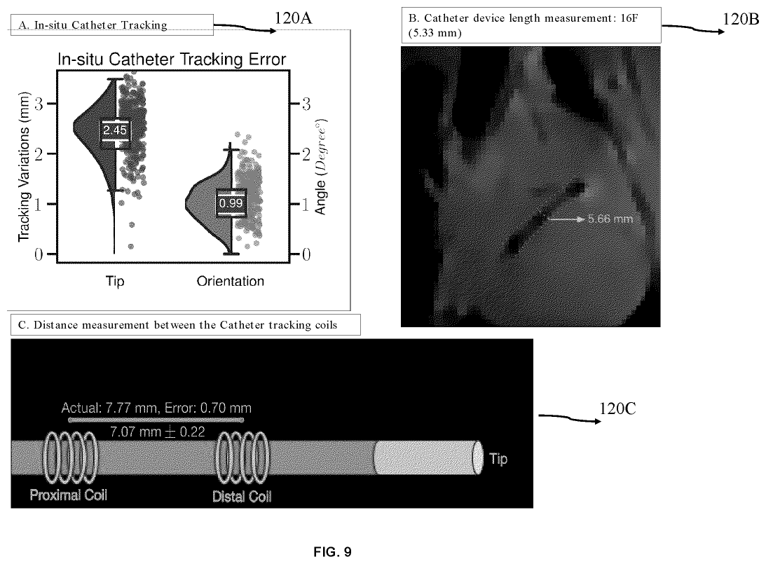

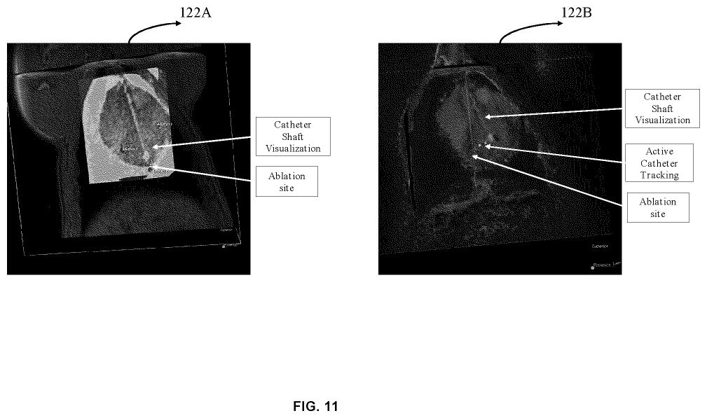

Acquiring ground-truth data in an in-situ model is more difficult, so we assess the precision of catheter tracking by quantifying changes in catheter orientation for a stationary catheter in-situ FIG. 9 120A. The orientation of the catheter is within small variations (give a degree or percent value), which indicates stable catheter tracking in-vivo. The width of the catheter device was measured with the distance between the individual tracking coils within a catheter shown in FIG. 9 120B and 120C. The device shaft visualization of the 2 catheters inside a porcine's heart is shown in FIG. 10 in sagittal view 121A, axial view 121B and coronal view 121C. For in-vivo healthy pig the targeted ablation and the relative position of the device shaft with the tracked catheter position is shown in FIG. 11 122A and 122B.

### Experiment 3: Human Study

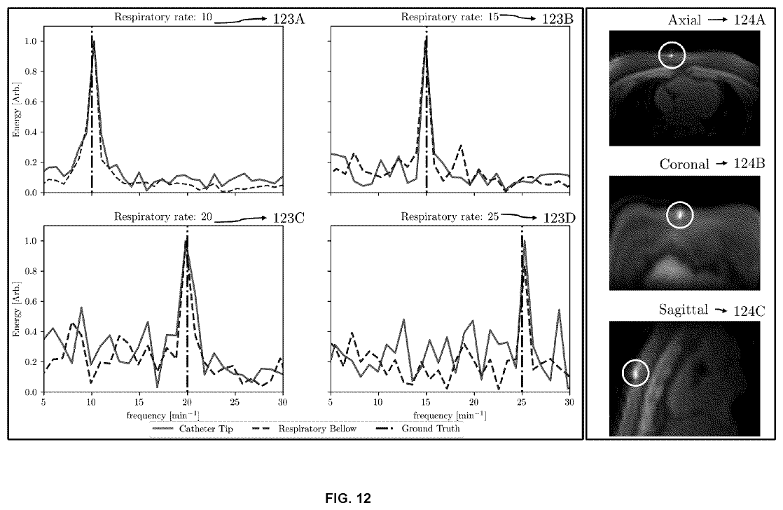

One human volunteer (M, 24y) was scanned with an MR-compatible ablation catheter taped to their chest. The volunteer was instructed to breathe along with a metronome while a cardiac-gated 3D cones acquisition was performed. A range of respiratory rates between 10 and 25 breaths/minute was used; metronome instructions were given at four times the respiratory rate with an audible click at beat 1, and two beats were used for inspiration and expiration stages. The result shows accurate tracking of the device with use of time-varying gradient trajectories in vivo respiratory breathing in FIG. 12, for respiratory rate of 10: 123A, respiratory rate of 15: 123B, respiratory rate of 20: 123C and respiratory rate of 25: 123D. The image of the undersampled anatomy with the device tracking coil bright signal is shown in FIG. 12, 124A, 124B and 124C in axial, coronal and sagittal, respectively.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

1. A method of tracking an interventional device during a magnetic resonance imaging procedure, the method comprising:
(a) employing a set of time-varying 3D non-Cartesian k-space trajectories to collect an undersampled magnetic resonance imaging dataset;
(b) processing the undersampled magnetic resonance imaging dataset to detect a local high-signal region having a known location on or within the interventional device; and
(c) employing the detected local high-signal region and the known location to provide feedback for tracking a position of the interventional device.

2. The method according to claim 1 wherein at least a portion of the undersampled magnetic resonance imaging dataset is collected using a set of imaging coils, the method further comprising processing the portion of the undersampled magnetic resonance imaging dataset collected with the set of imaging coils to generate an anatomical image.

3. The method according to claim 2 further comprising employing tracked position of the interventional device to perform motion correction on the anatomical image.

4. The method according to claim 1 further comprising, after having performed steps (a)-(c) a plurality of times, thereby obtaining a set of undersampled magnetic resonance imaging datasets;
(d) processing the set of undersampled magnetic resonance imaging datasets to generate an anatomical image.

5. The method according to claim 4 further comprising employing the tracked position of the interventional device to perform motion correction on the anatomical image.

6. The method according to claim 4 further comprising performing step (d) one or more times, such that the anatomical image is updated less frequently than the feedback associated with the tracked position of the interventional device.

7. The method according to claim 4 further comprising employing the set of time-varying 3D non-Cartesian k-space trajectories to generate visualization of the interventional device.

8. The method according to claim 7 further comprising generating a composite image comprising the anatomical image and the visualization.

9. The method according to claim 4 wherein the set of time-varying 3D non-Cartesian k-space trajectories fully sample k-space.

10. The method according claim 1 wherein the interventional device comprises a catheter.

11. The method according to claim 1 wherein each set of time-varying 3D non-Cartesian k-space trajectories is collected at a different cardiac cycle.
